(19) United States
(12) Patent Application Publication
(10) Pub. No.: US 2004/0121348 A1
(43) Pub. Date: Jun. 24, 2004

(54) Compositions and methods for treating pancreatic cancer

(75) Inventors: Roland Kreutzer, Weidenberg (DE); Stefan Limmer, Neudrossenfeld (DE); Hans-Peter Vornlocher, Bayreuth (DE); Anke Geick, Bayreuth (DE); Matthias Ocker, Erlangen (DE); Christoph Herold, Erlangen (DE); Detlef Schuppan, Bubenreuth (DE)

Correspondence Address: PALMER & DODGE, LLP, KATHLEEN M. WILLIAMS, 111 HUNTINGTON AVENUE, BOSTON, MA, 02199, US

(73) Assignee: Ribopharma AG

(21) Appl. No.: 10/384,434

(22) Filed: Mar. 7, 2003

Related U.S. Application Data
(63) Continuation-in-part of application No. PCT/EP02/11970, filed on Oct. 25, 2002.

(30) Foreign Application Priority Data
Oct. 26, 2001 (DE) 101 55 280.7
Nov. 29, 2001 (DE) 101 58 411.3
Dec. 7, 2001 (DE) 101 60 151.4
Jul. 9, 2002 (DE) 102 30 996.5
Jan. 9, 2002 (WO) PCT/EP02/00152
Jan. 9, 2002 (WO) PCT/EP02/00151
Oct. 25, 2002 (WO) PCT/EP02/11970

(51) Int. Cl. C12Q 1/68; G01N 33/574; C07H 21/04; C12N 9/64

(52) U.S. Cl. 435/6; 435/7.23; 435/69.1; 435/226; 435/320.1; 435/325; 536/23.2

(57) ABSTRACT

The present invention relates to a double-stranded ribonucleic acid (dsRNA) for inhibiting the expression of a K-ras oncogene, comprising a complementary RNA strand which is substantially identical to at least a part of a K-ras gene. The invention also relates to a pharmaceutical composition comprising the dsRNA together with a pharmaceutically acceptable carrier; methods for treating diseases caused by the expression of a K-ras oncogene using the pharmaceutical composition; and methods for inhibiting the expression of a K-ras oncogene in a cell.

### RELATED APPLICATIONS

This application is a continuation-in-part of International Application No. PCT/EP02/11970, which designated the United States and was filed on Oct. 25, 2002, which claims the benefit of German Patent No. 101 55 280.7, filed on Oct. 26, 2001, German Patent No 101 58 411.3, filed on Nov. 29, 2001, German Patent No. 101 60 151.4, filed on Dec. 7, 2001, EP Patent No. PCT/EP02/00152, filed on Jan. 9, 2002, EP Patent No. PCT/EP02/00151, filed Jan. 9, 2002, and German Patent No. 102 30 996.5, filed on Jul. 9, 2002. The entire teachings of the above application(s) are incorporated herein by reference.

### FIELD OF THE INVENTION

This invention relates to compositions and methods for treating pancreatic cancer using double-stranded ribonucleic acid (dsRNA) to inhibit the expression of an oncogene, such as a K-ras oncogene.

### BACKGROUND OF THE INVENTION

Many genetic diseases and defects are caused by only a minor mutation in a specific gene, such as a single point mutation (see, e.g., Cooper, D. N., et al., in “The Metabolic and Molecular Bases of Inherited Disease” (Scriver, C. R., et al., eds., (McGraw-Hill Inc., New York, Vol. 1, pp. 259-291 (1995)). For example, many forms of cancer are now known to be the result of the expression of “oncogenes.” Oncogenes are genetically altered genes whose altered expression product somehow disrupts normal cellular function or control. Most oncogenes are “activated” as the result of a mutation, often a point mutation, in the coding region of a normal cellular gene or of a “proto-oncogene.” Activation results in amino acid substitutions in the protein expression product, which, in turn, triggers a neoplastic transformation (Bishop, Cell (1991) 64:235-248). The underlying mutations can arise by various means, such as by chemical mutagenesis or ionizing radiation.

Activated cellular oncogenes are implicated in a variety of human tumors, including human bladder, colon, lung and mammary carcinoma cell lines (see, e.g., Cooper, et el., 1982; Krontiris, et al., 1981; Murray, et al., 1981; Perucho, et al., 1981), promyelocytic leukemia (Murray, et al., 1981), neuroblastoma (Shimizu, et al., 1983) and sarcoma cell lines (Pulciani, et al., 1982), and various solid tumors including carcinomas of the lung, and pancreas (Pulciani, et al., 1982). The ras oncogene family has been perhaps the best characterized to date (Barbacid, 1987; Bos, 1989). Most of the identified transforming genes in human carcinomas have been a member of the ras gene family, which encode immunologically related proteins having a molecular weight of 21,000 (p21) (Ellis, et al., 1981; Papageorge, et al., 1982). The ras family comprises at least three members, one transduces as H-ras in the Harvey strain of murine sarcoma virus (Ellis, et al., 1981), one identified by low stringency hybridization to H-ras, termed N-ras (Shimizu, et al., 1983), and one as K-ras and Kirsten murine sarcoma virus (Ellis, et al., 1981).

Pancreatic carcinoma and adenocarcinoma are among the carcinomas with the poorest prognosis. To date there is no adequate treatment. Although the exact cause is unknown, pancreatic carcinoma cells frequently have a mutation in the K-ras gene, particularly in codons 12, 13, and 61. The K-ras protein, an inner membrane associated protein, plays a key role in signal transduction (Lowy & Willumsen, Annu. Rev. Biochem. (1993) 62:851-891). Specifically, the Ras:GDP complex receives a signal from an upstream element (i.e., an activated membrane bound receptor) and the GDP is exchanged for GTP, thereby converting the inactive Ras:GDP complex to the active Ras:GTP complex. (Downward, et al., Proc. Natl. Acad. Sci. USA (1990) 87:5998-6002). In the oncogenic mutant forms, GAP induces GTP hydrolysis in the active Ras:GTP complex much more slowly, and thus the mutant forms remain in the active GTP form much longer than the wild-type (p21) protein (Gibbs, et al., Proc. Natl. Sci. USA (1998) 85:5026-5030). Presumably, the oncogenic properties of the mutant forms result from this extended transmission of signal. Thus, inhibiting RAS expression is believed to be a promising approach to the treatment and/or prevention of malignancies, such as cancer and other hyperproliferative conditions.

Double-stranded RNA molecules (dsRNA) have been shown to block gene expression in a highly conserved regulatory mechanism known as RNA interference (RNAi). Briefly, the RNAse III Dicer processes dsRNA into small interfering RNAs (siRNA) of approximately 22 nucleotides, which serve as guide sequences to induce target-specific mRNA cleavage by an RNA-induced silencing complex RISC (Hammond, S. M., et al., Nature (2000) 404:293-296). When administered to a cell or organism, exogenous dsRNA has been shown to direct the sequence-specific degradation of endogenous messenger RNA (mRNA) through RNAi. This phenomenon has been observed in a variety of organism, including mammals (see, e.g., WO 00/44895, Limmer; and DE 101 00 586 C1, Kruetzer et al.).

Thus, there is a need for an agent that can selectively and efficiently inhibit the expression of an activated oncogene, such as a RAS mutant gene, without also affecting the expression of the normal cellular gene or proto-oncogene. Such an agent would be useful for treating or preventing a number of malignancies, such as cancer and other hyperproliferative conditions.

### SUMMARY OF THE INVENTION

The present invention discloses double-stranded ribonucleic acid (dsRNA), as well as compositions and methods for inhibiting the expression of a K-ras oncogene in a cell using the dsRNA. The present invention also discloses compositions and methods for treating diseases caused by the expression of a K-ras oncogene. The dsRNA of the invention comprises an RNA strand (the complementary strand) having a region which is complementary to at least a portion of an RNA transcript of a K-ras oncogene.

In one aspect, the invention relates to double-stranded ribonucleic acid (dsRNA) for inhibiting the expression of a K-ras oncogene in a cell. The dsRNA comprises a complementary RNA strand having a nucleotide sequence which is complementary to at least a part of the K-ras oncogene and a second (sense) RNA strand, one of which strands may comprise a nucleotide overhang of 1 to 4, preferably 2 or 3, nucleotides in length. The nucleotide overhang may be on the 3′-terminus of the complementary RNA strand, and the 5′-end of the RNA strand may be blunt. The K-ras oncogene may be a K-ras gene having a point mutation in codon 12, which encodes an arginine, serine, alanine, valine, cystein, or asparagine; a point mutation in codon 13, which encodes an asparagine; or a point mutation in codon 61, which encodes histidine or leucine. The nucleotide sequence may be less than 25 nucleotides in length, 19 to 24 nucleotides in length, 20 to 24 nucleotides in length, 21 to 23 nucleotides in length, or 22 or 23 nucleotides in length. The complementary RNA strand may be less than 30 nucleotides in length, less 25 nucleotides in length, or 21 to 24 nucleotides in length. The dsRNA may further comprise a second (sense) RNA strand. The complementary RNA strand may be 23 nucleotides in length and the second RNA strand may be 21 nucleotides in length. The complementary RNA strand may have a 3′-end and a 5′-end, wherein the 3′-end has a nucleotide overhang of 2 nucleotides in length and the 5′-end is blunt. The nucleotide sequence of the complementary RNA strand may be complementary to a primary or processed RNA transcript of the K-ras oncogene. The complementary RNA strand may comprise SEQ ID NO:2 and the second RNA strand may comprise SEQ ID NO: 1; the complementary RNA strand may comprise SEQ ID NO:4 and the second RNA strand may comprise SEQ ID NO:3; or the complementary RNA strand may be comprise SEQ ID NO:5 and the second RNA strand may comprise SEQ ID NO:6. The cell may be a pancreatic carcinoma cell.

In another aspect, the invention relates to a method for inhibiting the expression of a K-ras oncogene in a cell. The method comprises introducing a dsRNA, as described above, into the cell, and maintaining the cell for a time sufficient to obtain degradation of a mRNA transcript of the K-ras oncogene. The cell may be a pancreatic carcinoma cell.

In yet another aspect, the invention relates to a pharmaceutical composition for inhibiting the expression of a K-ras oncogene in an organism. The compositions comprises a dsRNA, as described above, and a pharmaceutically acceptable carrier. The cell organism may be a mammal, such as a human. The dosage unit of dsRNA may be less than 5 milligram (mg) of dsRNA per kg body weight of the mammal, in a range of 0.01 to 2.5 milligrams (mg), 0.1 to 200 micrograms (μg), 0.1 to 100 μg per kilogram body weight of the mammal, or less than 25 μg per kilogram body weight of the mammal. The pharmaceutically acceptable carrier may be an aqueous solution, such as phosphate buffered saline, or it may comprise a micellar structure, such as a liposome, capsid, capsoid, polymeric nanocapsule, or polymeric microcapsule.

In still another aspect, the invention relates to a method for treating a disease caused by the expression of a K-ras oncogene in a mammal. The method comprises administering a pharmaceutical composition comprising a dsRNA, as described above, and a pharmaceutically acceptable carrier.

The details of once or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 shows the apoptosis rate (percentage) of human YAP C pancreatic carcinoma cells after transfection with a dsRNA comprising a nucleotide sequence complementary to a first sequence of the human K-ras gene.

FIG. 2 shows the number of living cells at various times post-transfection with a dsRNA comprising a nucleotide sequence complementary to a first sequence of the human K-ras gene.

FIG. 3 shows the volume of subcutaneously implanted human pancreatic adenocarcinoma in NMRI mice.

### DETAILED DESCRIPTION OF THE INVENTION

The present invention discloses double-stranded ribonucleic acid (dsRNA), as well as compositions and methods for inhibiting the expression of a K-ras oncogene in a cell using the dsRNA. The present invention also discloses compositions and methods for treating diseases caused by the expression of a K-ras oncogene. The dsRNA of the invention comprises an RNA strand (the complementary strand) having a region which is complementary to at least a portion of an RNA transcript of a K-ras oncogene.

The dsRNA of the invention comprises an RNA strand (the complementary strand) which is complementary to at least a portion of an RNA transcript of an oncogene, such as a K-ras oncogene. The use of these dsRNAs enables the targeted degradation of mRNAs of genes that are implicated in uncontrolled cell or tissue growth. Using cell-based assays, the present inventors have demonstrated that very low dosages of these dsRNA can specifically and efficiently mediate RNAi, resulting in significant inhibition of expression of the target gene. dsRNA affects apoptosis to such an extent that there is a noticeable reduction in both tumor size and number of tumor cells. Thus, the present invention encompasses these dsRNAs and compositions comprising dsRNA and their use for specifically silencing oncogenes whose protein products are implicated in abnormal cell growth and malignant transformations. Moreover, the dsRNAs of the invention have no apparent effect on neighboring normal cells. Thus, the methods and compositions of the present invention comprising these dsRNAs are useful for treating cellular proliferative and/or differentiation disorders, such as cancer.

The following detailed description discloses how to make and use the dsRNA and compositions containing dsRNA to inhibit the expression of K-ras oncogenes, as well as compositions and methods for treating diseases and disorders caused by the expression of these genes. The pharmaceutical compositions of the present invention comprise a dsRNA having an RNA strand comprising a complementary region which is complementary to at least a portion of an RNA transcript of a K-ras oncogene, together with a pharmaceutically acceptable carrier. The K-ras oncogene may be any mutant form or variation of the wild-type K-ras gene.

Accordingly, certain aspects of the present invention relate to pharmaceutical compositions comprising the dsRNA of the present invention together with a pharmaceutically acceptable carrier, methods of using the compositions to inhibit expression of a target K-ras oncogene, and methods of using the pharmaceutical compositions to treat diseases caused by the expression of a K-ras oncogene.

I. Definitions

For convenience, the meaning of certain terms and phrases used in the specification, examples, and appended claims, are provided below.

As used herein, “target gene” refers to a section of a DNA strand of a double-stranded DNA that is complementary to a section of a DNA strand, including all transcribed regions, that serves as a matrix for transcription. A target gene, usually the sense strand, is a gene whose expression is to be selectively inhibited or silenced through RNA interference. As used herein, the term “target gene” specifically encompasses any cellular gene or gene fragment whose expression or activity is associated with abnormal cellular proliferation or malignant transformation.

As used herein, the term “oncogene” refers to a gene whose product is involved either in transforming cells in culture or in inducing cancer in animals. “Proto-oncogene” refers to a normal gene involved in the control of cell growth or division. As used herein, an oncogene is a mutant form of a proto-oncogene.

The term “complementary RNA strand” (also referred to herein as the “antisense strand”) refers to the strand of a dsRNA which is complementary to an mRNA transcript that is formed during expression of the target gene, or its processing products. As used herein, the term “complementary nucleotide sequence” refers to the region on the complementary RNA strand that is complementary to an mRNA transcript of a portion of the target gene. “dsRNA” refers to a ribonucleic acid molecule having a duplex structure comprising two complementary and anti-parallel nucleic acid strands. Not all nucleotides of a dsRNA must exhibit Watson-Crick base pairs; the two RNA strands may be substantially complementary (i.e., having no more than one or two nucleotide mismatches). The maximum number of base pairs is the number of nucleotides in the shortest strand of the dsRNA. The RNA strands may have the same or a different number of nucleotides. The dsRNA is less than 30, preferably less than 25, more preferably 21 to 24, and most preferably 23 nucleotides in length. dsRNAs of this length are particularly efficient in inhibiting the expression of the target K-ras oncogene. “Introducing into” means uptake or absorption in the cell, as is understood by those skilled in the art. Absorption or uptake of dsRNA can occur through cellular processes, or by auxiliary agents or devices. For example, for in vivo delivery, dsRNA can be injected into a tissue site or administered systemically. In vitro delivery includes methods known in the art such as electroporation and lipofection.

As used herein, a “nucleotide overhang” refers to the unpaired nucleotide or nucleotides that protrude from the duplex structure when a 3′-end of one RNA strand extends beyond the 5′-end of the other strand, or vice versa.

As used herein and as known in the art, the term “identity” is the relationship between two or more polynucleotide sequences, as determined by comparing the sequences. Identity also means the degree of sequence relatedness between polynucleotide sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While there exist a number of methods to measure identity between two polynucleotide sequences, the term is well known to skilled artisans (see, e.g., Sequence Analysis in Molecular Biology, von Heinje, G., Academic Press (1987); and Sequence Analysis Primer, Gribskov., M. and Devereux, J., eds., M. Stockton Press, New York (1991)). Methods commonly employed to determine identity between sequences include, for example, those disclosed in Carillo, H., and Lipman, D., SIAM J. Applied Math. (1988) 48:1073. “Substantially identical,” as used herein, means there is a very high degree of homology (preferably 100% sequence identity) between the sense strand of the dsRNA and the corresponding part of the target gene. However, dsRNA having greater than 90%, or 95% sequence identity may be used in the present invention, and thus sequence variations that might be expected due to genetic mutation, strain polymorphism, or evolutionary divergence can be tolerated. Although 100% identity is preferred, the dsRNA may contain single or multiple base-pair random mismatches between the RNA and the target gene.

As used herein, the term “treatment” refers to the application or administration of a therapeutic agent to a patient, or application or administration of a therapeutic agent to an isolated tissue or cell line from a patient, who has a disorder, e.g., a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a dsRNA molecule has been introduced by means of recombinant DNA techniques.

II. Double-Stranded Ribonucleic Acid (dsRNA)

In one embodiment, the invention relates to a double-stranded ribonucleic acid (dsRNA) having a nucleotide sequence which is substantially identical to at least a portion of a mutant form of a K-ras gene, i.e., a K-ras oncogene. The dsRNA comprises two RNA strands that are sufficiently complementary to hybridize to form the duplex structure. One strand of the dsRNA comprises the nucleotide sequence that is substantially identical to a portion of the target gene (the “sense” strand), and the other strand (the “complementary” or “antisense” strand) comprises a sequence that is complementary to an RNA transcript of the target K-ras oncogene. The complementary region is less than 25 nucleotides, preferably 19 to 24 nucleotides, more preferably 20 to 24 nucleotides, even more preferably 21 to 23 nucleotides, and most preferably 22 or 23 nucleotides in length. The dsRNA is less than 30 nucleotides, preferably less than 25 nucleotides, and most preferably between 21 and 24 nucleotides in length. The dsRNA can be synthesized by standard methods known in the art, e.g., by use of an automated DNA synthesizer, such as are commercially available from Biosearch, Applied Biosystems, Inc. In one embodiment, the target gene is a K-ras gene having a mutation in at least one of codons 12, 13 and/or 61. In a preferred embodiment, the target oncogene is a K-ras gene having a mutation at codon 12 that codes for arginine, serine, alanine, valine, cystein, or asparagine, rather than the wild-type glycine. In another preferred embodment, the target oncogene is a K-ras gene having a mutation at codon 13 that codes for asparagine, rather than the wild-type glycines. In yet another preferred embodiment, the target oncogene is a K-ras gene having a mutation at codon 61 that codes for histidine or leucine, rather than the wild-type glutamine. In specific embodiments, the complementary (antisense) RNA strand of the dsRNA comprises the sequence set forth in SEQ ID NO:2 and the second (sense) RNA strand comprises the sequence set forth in SEQ ID NO:1; or the complementary (antisense) RNA strand of the dsRNA comprises the sequence set forth in SEQ ID NO:4 and the second (sense) RNA strand comprises the sequence set forth in SEQ ID NO:3; or the complementary (antisense) RNA strand of the dsRNA comprises the sequence set forth in SEQ ID NO:6 and the second (sense) RNA strand comprises the sequence set forth in SEQ ID NO:5.

In one embodiment, at least one end of the dsRNA has a single-stranded nucleotide overhang of 1 to 4, preferably 2 or 3 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without effecting its overall stability. dsRNA having only one overhang has proven particularly stable and effective in vivo, as well as in a variety of cells, cell culture mediums, blood, and serum. Preferably, the single-stranded overhang is located at the 3′-terminal end of the complementary (antisense) RNA strand or, alternatively, at the 3′-terminal end of the second (sense) strand. The dsRNA may also have a blunt end, preferably located at the 5′-end of the complementary (antisense) strand. Such dsRNAs have improved stability and inhibitory activity, thus allowing administration at low dosages, i.e., less than 5 mg/kg body weight of the recipient per day. Preferably, the complementary strand of the dsRNA has a nucleotide overhang at the 3′-end, and the 5′-end is blunt. In a particularly preferred embodiment, the complementary RNA strand is 23 nucleotides in length, the sense RNA strand is 21 nucleotides in length, and 3′-end of the complementary RNA strand comprises a 1 or 2-nucleotide overhang, and the 5′-end is blunt.

III. Pharmaceutical Compositions Comprising dsRNA

In one embodiment, the invention relates to a pharmaceutical composition comprising a dsRNA, as described in the preceding section, and a pharmaceutically acceptable carrier, as described below. The pharmaceutical composition comprising the dsRNA is useful for treating a disease or disorder associated with the expression or activity of a K-ras oncogene.

The pharmaceutical compositions of the present invention are administered in dosages sufficient to inhibit expression of the target gene. The present inventors have found that, because of their improved efficiency, compositions comprising the dsRNA of the invention can be administered at surprisingly low dosages. A maximum dosage of 5 mg dsRNA per kilogram body weight of recipient per day is sufficient to inhibit or completely suppress expression of the target gene.

In general, a suitable dose of dsRNA will be in the range of 0.01 to 5.0 milligrams per kilogram body weight of the recipient per day, preferably in the range of 0.1 to 2.5 milligrams per kilogram body weight per day, more preferably in the range of 0.1 to 100 micrograms per kilogram body weight per day, more preferably in the range of 0.1 to 200 micrograms per kilogram body weight per day, even more preferably in the range of 1.0 to 50 micrograms per kilogram body weight per day, and most preferably in the range of 1.0 to 25 micrograms per kilogram body weight per day. The pharmaceutical composition may be administered once daily, or the dsRNA may be administered as two, three, four, five, six or more sub-doses at appropriate intervals throughout the day. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

Advances in mouse genetics have generated a number of mouse models for the study of various human diseases. For example, mouse models are available for hematopoietic malignancies such as leukemias, lymphomas and acute myelogenous leukemia. The MMHCC (Mouse models of Human Cancer Consortium) web page (emice.nci.nih.gov), sponsored by the National Cancer Institute, provides disease-site-specific compendium of known cancer models, and has links to the searchable Cancer Models Database (cancermodels.nci.nih.gov), as well as the NCI-MMHCC mouse repository. Examples of the genetic tools that are currently available for the modeling of leukemia and lymphomas in mice, and which are useful in practicing the present invention, are described in the following references: Maru, Y., Int. J. Hematol. (2001) 73:308-322; Pandolfi, P. P., Oncogene (2001) 20:5726-5735; Pollock, J. L., et al., Curr. Opin. Hematol. (2001) 8:206-211; Rego, E. M., et al., Semin. in Hemat. (2001) 38:4-70; Shannon, K. M., et al. (2001) Modeling myeloid leukemia tumors suppressor gene inactivation in the mouse, Semin. Cancer Biol. 11, 191-200; Van Etten, R. A., (2001) Curr. Opin. Hematol. 8, 224-230; Wong, S., et al. (2001) Oncogene 20, 5644-5659; Phillips J A., Cancer Res. (2000) 52(2):437-43; Harris, A. W., et al, J. Exp. Med. (1988) 167(2):353-71; Zeng X X et al., Blood. (1988) 92(10):3529-36; Eriksson, B., et al., Exp. Hematol. (1999) 27(4):682-8; and Kovalchuk, A., et al., J. Exp. Med. (2000) 192(8):1183-90. Mouse repositories can also be found at: The Jackson Laboratory, Charles River Laboratories, Taconic, Harlan, Mutant Mouse Regional Resource Centers (MMRRC) National Network and at the European Mouse Mutant Archive. Such models may be used for in vivo testing of dsRNA, as well as for determining a therapeutically effective dose.

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the pharmaceutical compositions are administered by intravenous or intraparenteral infusion or injection.

For oral administration, the dsRNAs useful in the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of dsRNA in the cells that express the target gene. Such substances include, for example, micellar structures, such as liposomes or capsids, as described below. Surprisingly, the present inventors have discovered that compositions containing only naked dsRNA and a physiologically acceptable solvent are taken up by cells, where the dsRNA effectively inhibits expression of the target gene. Although microinjection, lipofection, viruses, viroids, capsids, capsoids, or other auxiliary agents are required to introduce dsRNA into cell cultures, surprisingly these methods and agents are not necessary for uptake of dsRNA in vivo. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

In one embodiment, the encapsulated formulation comprises a viral coat protein. In this embodiment, the dsRNA may be bound to, associated with, or enclosed by at least one viral coat protein. The viral coat protein may be derived from or associated with a virus, such as a polyoma virus, or it may be partially or entirely artificial. For example, the coat protein may be a Virus Protein 1 and/or Virus Protein 2 of the polyoma virus, or a derivative thereof.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in formulation a range of dosage for use in humans. The dosage of compositions of the invention lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, the dsRNAs useful according to the invention can be administered in combination with other known agents effective in treatment of diseases. In any event, the administering physician can adjust the amount and timing of dsRNA administration on the basis of results observed using standard measures of efficacy known in the art or described herein.

For oral administration, the dsRNAs useful in the invention will generally be provided in the form of tablets or capsules, as a powder or granules, or as an aqueous solution or suspension.

IV. Methods for Treating Diseases Caused by Expression of an K-ras Oncogene.

In one embodiment, the invention relates to a method for treating a subject having a disease or at risk of developing a disease caused by the expression of a K-ras oncogene. In this embodiment, the dsRNA can act as novel therapeutic agents for controlling one or more of cellular proliferative and/or differentiative disorders. The method comprises administering a pharmaceutical composition of the invention to the patient (e.g., human), such that expression of the target gene is silenced. Because of their high specificity, the dsRNAs of the present invention specifically target mRNAs of target genes of diseased cells and tissues, as described below, and at surprisingly low dosages. In a preferred embodiment, the disease to be treated is a pancreatic carcinoma.

In the prevention of disease, the target gene may be one which is required for initiation or maintenance of the disease, or which has been identified as being associated with a higher risk of contracting the disease. In the treatment of disease, the dsRNA can be brought into contact with the cells or tissue exhibiting the disease. For example, dsRNA substantially identical to all or part of a mutated gene associated with cancer, or one expressed at high levels in tumor cells, e.g. aurora kinase, may be brought into contact with or introduced into a cancerous cell or tumor gene.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of pancreas, prostate, colon, lung, breast and liver origin. As used herein, the terms “cancer,” “hyperproliferative,” and “neoplastic” refer to cells having the capacity for autonomous growth, i.e., an abnormal state of condition characterized by rapidly proliferating cell growth. These terms are meant to include all types of cancerous growths or oncogenic processes, metastatic tissues or malignantly transformed cells, tissues, or organs, irrespective of histopathologic type or stage of invasiveness. Proliferative disorders also include hematopoietic neoplastic disorders, including diseases involving hyperplastic/neoplatic cells of hematopoietic origin, e.g., arising from myeloid, lymphoid or erythroid lineages, or precursor cells thereof.

In addition to the above-described K-ras oncogenes, other genes can be targeted for treatment including, without limitation, other oncogenes (see, e.g., Hanahan, D. and R. A. Weinberg, Cell (2000) 100:57; and Yokota, J., Carcinogenesis (2000) 21(3):497-503); genes of proteins that are involved in metastasizing and/or invasive processes (Boyd, D., Cancer Metastasis Rev. (1996) 15(1):77-89; Yokota, J., Carcinogenesis (2000) 21(3):497-503); genes of proteases as well as of molecules that regulate apoptosis and the cell cycle (Matrisian, L. M., Curr. Biol. (1999) 9(20):R776-8; Krepela, E., Neoplasma (2001) 48(5):332-49; Basbaum and Werb, Curr. Opin. Cell Biol. (1996) 8:731-738; Birkedal-Hansen, et al., Crit. Rev. Oral Biol. Med. (1993) 4:197-250; Mignatti and Rifkin, Physiol. Rev. (1993) 73:161-195; Stetler-Stevenson, et al., Annu. Rev. Cell Biol. (1993) 9:541-573; Brinkerhoff, E., and L. M. Matrisan, Nature Reviews (2002) 3:207-214; Strasser, A., et al., Annu. Rev. Biochem. (2000) 69:217-45; Chao, D. T. and S. J. Korsmeyer, Annu. Rev. Immunol. (1998) 16:395-419; Mullauer, L., et al., Mutat. Res. (2001) 488(3):211-31; Fotedar, R., et al., Prog. Cell Cycle Res. (1996) 2:147-63; Reed, J. C., Am. J. Pathol. (2000) 157(5):1415-30; D'Ari, R., Bioassays (2001) 23(7):563-5); genes that express the EGF receptor; Mendelsohn, J. and J. Baselga, Oncogene (2000) 19(56):6550-65; Normanno, N., et al., Front. Biosci. (2001) 6:D685-707); and the multi-drug resistance 1 gene, MDR1 gene (Childs, S., and V. Ling, Imp. Adv. Oncol. (1994) 21-36).

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the pharmaceutical compositions are administered by intravenous or intraparenteral infusion or injection.

V. Methods for Inhibiting Expression of an K-ras Oncogene

In yet another aspect, the invention relates to a method for inhibiting the expression of a K-ras oncogene in a mammal. The method comprises administering a composition of the invention to the mammal such that expression of the target K-ras oncogene is silenced. Because of their high specificity, the dsRNAs of the present invention specifically target RNAs (primary or processed) of target K-ras oncogenes, and at surprisingly low dosages. Compositions and methods for inhibiting the expression of these target genes using dsRNAs can be performed as described elsewhere herein.

In one embodiment, the invention comprises administering a composition comprising a dsRNA, wherein the dsRNA comprises a nucleotide sequence which is complementary to at least a part of an RNA transcript of the target K-ras oncogene of the mammal (e.g., human) to be treated. The composition may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the compositions are administered by intravenous or intraparenteral infusion or injection.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Inhibition of Kras Gene Expression by RNA Interference

Synthesis and Preparation of dsRNAs

Oligoribonucleotides are synthesized with an RNA synthesizer (Expedite 8909, Applied Biosystems, Weiterstadt, Germany) and purified by High Pressure Liquid Chromatography (HPLC) using NucleoPac PA-100 columns, 9×250 mm (Dionex Corp.; low salt buffer: 20 mM Tris, 10 mM NaClO₄, pH 6.8, 10% acetonitrile; the high-salt buffer was: 20 mM Tris, 400 mM NaClO4, pH 6.8, 10% acetonitrile. flow rate: 3 ml/min). Formation of double stranded siRNAs is then achieved by heating a stoichiometric mixture of the individual complementary strands (10 μM) in 10 mM sodium phosphate buffer, pH 6.8, 100 mM NaCl, to 80-90° C., with subsequent slow cooling to room temperature over 6 hours,

In addition, dsRNA molecules with linkers may be produced by solid phase synthesis and addition of hexaethylene glycol as a non-nucleotide linker (D. Jeremy Williams, Kathleen B. Hall, Biochemistry, 1996, 35, 14665-14670). A Hexaethylene glycol linker phosphoramidite (Chruachem Ltd, Todd Campus, West of Scotland Science Park, Acre Road, Glasgow, G20 OUA, Scotland, UK) is coupled to the support bound oligoribonucleotide employing the same synthetic cycle as for standard nucleoside phosphoramidites (Proligo Biochemie GmbH, Georg-Hyken-Str.14, Hamburg, Germany) but with prolonged coupling times. Incorporation of linker phosphoramidite is comparable to the incorporation of nucleoside phosphoramidites.

The double-stranded oligoribonucleotides having the following sequences were synthesized (SEQ ID NO: 1 to SEQ ID NO: 8 in the sequence protocol):

KRAS1, which is complementary to a sequence of the human K-ras gene in YAP C cells that exhibits a first point mutation in codon 12:

S2: 5′-agu ugg agc ugu ugg cgu agg-3′ (SEQ ID NO: 1)

S1: 3′-ca uca acc ucg aca acc gca ucc-5′ (SEQ ID NO: 2)

KRAS1′, which is complementary to a sequence of the human K-ras gene in a human pancreatic adenocarcinoma implanted subcutaneously in NMRI mice that exhibits a first point mutation in codon 12:

S2: 5′-agu ugg age uga ugg cgu agg-3′ (SEQ ID NO: 3)

S1: 3′-ca uca acc ucg acu acc gea ucc-5′ (SEQ ID NO: 4)

KRAS2, which is complementary to the wild type sequence from the human K-ras gene:

S2: 5′-agu ugg age ugg ugg cgu agg-3′ (SEQ ID NO: 5)

S1.: 3′-ca uca acc ucg acc acc gea ucc-5′ (SEQ ID NO: 6)

NEO, which is complementary to sequence from the neomycin resistance gene:

S2: 5′-c aag gau gag gau cgu uuc gca-3′ (SEQ ID NO: 7)

S1: 3′-ucu guc cua cuc cua gea aag cg -5′ (SEQ ID NO: 8)

Cells from the human pancreatic carcinoma cell line YAP C, which were obtained from the German Collection of Microorganisms and Cell Cultures, Braunschweig (No. ACC 382) were cultured at 37° C., 5% CO₂ in RPMI 1640 medium (Biochrom, Berlin) with 10% fetal calf serum (FCS) and 100 μg/ml penicillin/streptomycin. Transfections were carried out in a 6-well plate with oligofectamine (Invitrogen, Karlsruhe). 150,000 cells were placed in each well. Double-stranded oligoribonucleotides were transfected into the cells according to the protocol recommended by Invitrogen for oligofectamine (the data relate to one well in a 6-well plate): 10 μl of double-stranded oligoribonucleotides (0.1 to 10 μM) were diluted with 175 μl cell culture medium without additives. 3 μl oligofectamine was diluted with 12 μl cell culture medium without additives and incubated for 10 minutes at room temperature. The oligofectamine diluted in this way was then added to the already diluted double-stranded oligoribonucleotides, mixed, and incubated for 20 minutes at room temperature. During this time, the cells to be transfected were washed once with cell culture medium without additives, and replenished with 800 μl of fresh cell culture medium. Then 200 μl of the described oligofectamine-dsRNA mixture were added to each well, so that the end transfection volume was 1000 μl. This resulted in an end concentration of double-stranded oligoribonucleotides of 1-100 nM. The transfection assay was incubated for 4 hours at 37° C. 500 μl cell culture medium with 30% FCS was then added to each well, so that the end concentration of FCS was 10%. This assay was then incubated for 24 to 120 hours at 37° C.

To determine the apoptosis rate, the supernatant fluid was collected after incubation, the cells were washed with phosphate buffered saline solution (PBS), trypsinized, and centrifuged for 10 minutes at 100 g. The supernatant fluid was then discarded, and the pellets were incubated in hypotonic propidium iodide solution in the dark for 30 minutes at 4° C. The pelletted cells were then analyzed by flow cytometry using a fluorescence-supported FACSCalibur cell sorter (BD GmbH, Heidelberg).

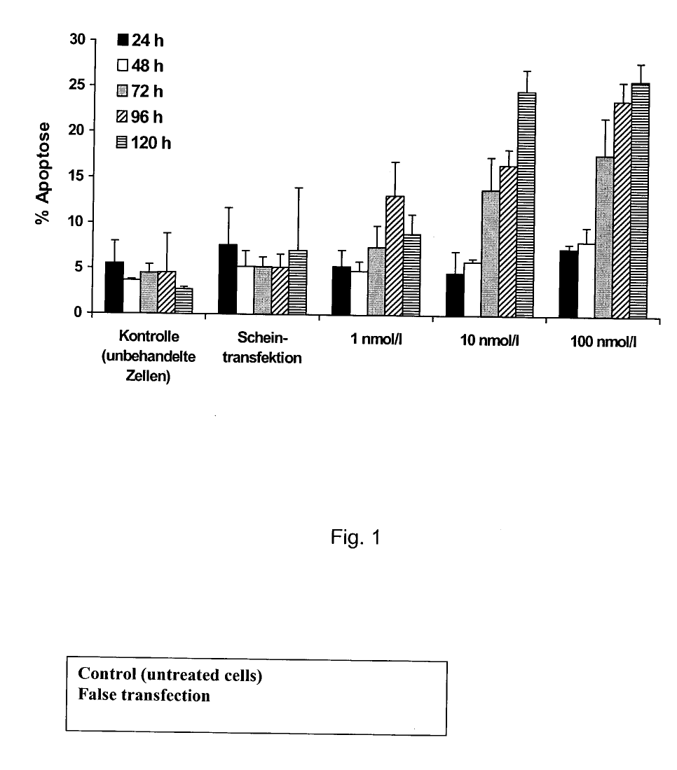

FIG. 1 shows the apoptosis rate (in percent) of human pancreatic YAP C carcinoma cells, dependent on incubation time after transfection with increasing concentrations of KRAS1 dsRNA. From this it may be seen that KRAS1 induces concentration-dependent apoptosis in human pancreatic carcinoma cells. The apoptosis rate increases with incubation time. Whereas untreated YAP C cells (control) and cells with which the described method of transfection was carried out without double-stranded oligoribonucleotides (mock- or false transfection) also only exhibit a maximum 5% apoptosis after 120 hours incubation, transfection with 100 nM KRAS1 increased the apoptosis rate after 120 hours to 24%. KRAS2 dsRNA that is complementary to the K-ras wild type induced apoptosis in YAP C cells with the same effectiveness.

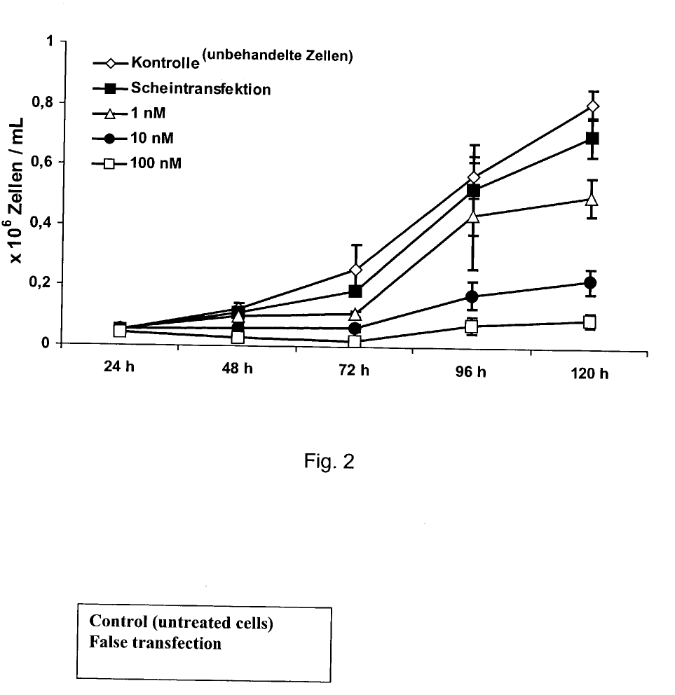

To determine the effect of transfection on proliferation and the number of live cells, respectively, 50,000 YAP C cells were added to each well in a 6-well plate and transfected as described above. The number of live cells was determined with trypan blue exclusion staining after 24 to 120 hours incubation time by counting in a Neubauer counting chamber. The results are shown in FIG. 2. The inhibition of YAP C cell proliferation by KRAS1 depended on concentration of dsRNA. The number of living cells was statistically significantly reduced using only 1 nM KRAS1 (p=0.001 in contrast to untreated controls after 120 hours).

Transfection with the KRAS2 dsRNA that is complementary to the K-ras wild type leads to a reduction in the number of live cells at a concentration of 100 nM. Non-malignant human skin fibroblasts showed no change in their proliferation behavior by being transfected with KRAS1 or KRAS2.

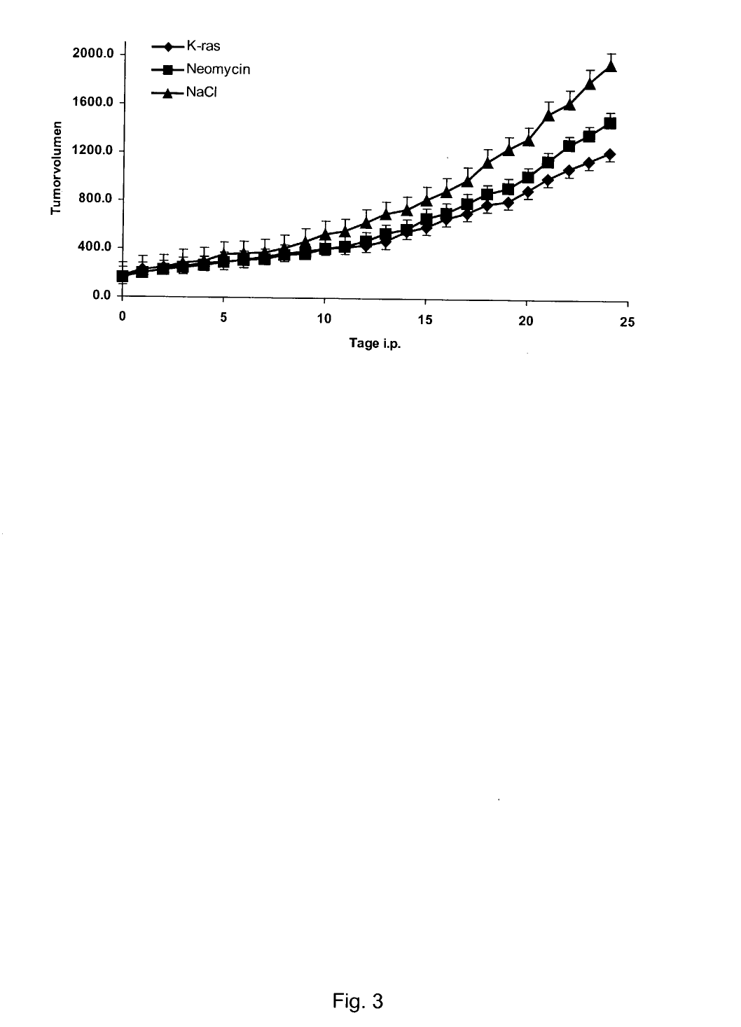

For the in vivo experiments, human pancreatic adenocarcinoma tissue fragments having a diameter of 2-3 mm were implanted subcutaneously in NMRI mice (Harlan Winkelmann GmbH, Borchen). After the tumors had grown to a size of 6-7 mm, 200 μg KRAS1′ or NEO per kg body weight, each dissolved in a physiological saline solution, were injected intraperitoneally. A physiological saline solution was injected as a control. The tumors were measured daily using a slide gauge or standardized template. FIG. 3 shows the measured tumor volumes in mm³ as an average value +/− standard error of the average value, dependent on the number of days since the start of treatment with intraperitoneal injection (days i.p.). dsRNA that is complementary to the K-ras gene was capable of inhibiting the growth of the tumors. The tumor was inhibited by daily intraperitoneal application of a dsRNA that is complementary to the K-ras gene, at a dosage of 200 μg/kg, such that the tumor volumes after 24 days of treatment were only 62% of the tumor volumes seen in the control group.

### Example 2

### Treatment of a Pancreatic Cancer Patient with Kras dsRNAs

In this Example, Kras dsRNAs are injected into a pancreatic cancer patient and shown to specifically inhibit K-Ras gene expression.

dsRNA Administration and Dosage

The present example provides for pharmaceutical compositions for the treatment of human pancreatic cancer patients comprising a therapeutically effective amount of a Kras dsRNAs as disclosed herein, in combination with a pharmaceutically acceptable carrier or excipient. K Ras dsRNAs useful according to the invention may be formulated for oral or parenteral administration. The pharmaceutical compositions may be administered in any effective, convenient manner including, for instance, administration by topical, oral, anal, vaginal, intravenous, intraperitoneal, intramuscular, subcutaneous, intranasal or intradermal routes among others. One of skill in the art can readily prepare dsRNAs for injection using such carriers that include, but are not limited to, saline, buffered saline, dextrose, water, glycerol, ethanol, and combinations thereof. Additional examples of suitable carriers are found in standard pharmaceutical texts, e.g. “Remington's Pharmaceutical Sciences”, 16th edition, Mack Publishing Company, Easton, Pa., 1980.

The dosage of the Kras dsRNAs will vary depending on the form of administration. In the case of an injection, the therapeutically effective dose of dsRNA per injection is in a dosage range of approximately 1-500 μg/kg body weight, preferably 100 μg/kg body weight. In addition to the active ingredient, the compositions usually also contain suitable buffers, for example phosphate buffer, to maintain an appropriate pH and sodium chloride, glucose or mannitol to make the solution isotonic. The administering physician will determine the daily dosage which will be most suitable for an individual and will vary with the age, gender, weight and response of the particular individual, as well as the severity of the patient's symptoms. The above dosages are exemplary of the average case. There can, of course, be individual instances where higher or lower dosage ranges are merited, and such are within the scope of this invention. The Kras dsRNAs of the present invention may be administered alone or with additional dsRNA species or in combination with other pharmaceuticals.

RNA Purification and Analysis

Efficacy of the KRas dsRNA treatment is determined at defined intervals after the initiation of treatment using real time PCR on total RNA extracted from tissue biopsies. Cytoplasmic RNA from tissue biopsies, taken prior to and during treatment, is purified with the help of the RNeasy Kit (Qiagen, Hilden) and K-Ras mRNA levels are quantitated by real time RT-PCR as described previously (Eder M et al. Leukemia 1999; 13: 1383-1389; Scherr M et al. BioTechniques. 2001; 31: 520-526). Analysis of K-Ras mRNA levels before and during treatment by real time PCR, provides the attending physician with a rapid and accurate assessment of treatment efficacy as well as the opportunity to modify the treatment regimen in response to the patient's symptoms and disease progression.

### Example 3

### K-Ras dsRNA Expression Vectors

In another aspect of the invention, K-Ras specific dsRNA molecules that interact with K-Ras target RNA molecules and modulate K-Ras gene expression activity are expressed from transcription units inserted into DNA or RNA vectors (see for example Couture et A, 1996, TIG., 12, 5 1 0, Skillern et A, International PCT Publication No. WO 00/22113, Conrad, International PCT Publication No. WO 00/22114, and Conrad, U.S. Pat. No. 6,054,299). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann et al., 1995, Proc. Natl. Acad. Sci. USA 92:1292).

The individual strands of a K-Ras dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant K-Ras dsRNA expression vectors are preferably DNA plasmids or viral vectors. K-Ras dsRNA expressing viral vectors can be constructed based on, but not limited to, adeno-associated virus (for a review, see Muzyczka et al. (1992, Curr. Topics in Micro and Immunol. 158:97-129)), adenovirus (see, for example, Berkner et al. (1988, BioTechniques 6:616), Rosenfeld et al. (1991, Science 252:431-434), and Rosenfeld et al. (1992, Cell 68:143-155)), or alphavirus as well as others known in the art. Retroviruses have been used to introduce a variety of genes into many different cell types, including epithelial cells, in vitro and/or in vivo (see for example Eglitis, et al., 1985, Science 230:1395-1398; Danos and Mulligan, 1988, Proc. NatI. Acad. Sci. USA 85:6460-6464; Wilson et al., 1988, Proc. NatI. Acad. Sci. USA 85:3014-3018; Armentano et al., 1990, Proc. NatI. Acad. Sci. USA 87:61416145; Huber et al., 1991, Proc. NatI. Acad. Sci. USA 88:8039-8043; Ferry et al., 1991, Proc. NatI. Acad. Sci. USA 88:8377-8381; Chowdhury et al., 1991, Science 254:1802-1805; van Beusechem. et al., 1992, Proc. Nad. Acad. Sci. USA 89:7640-19; Kay et al., 1992, Human Gene Therapy 3:641-647; Dai et al., 1992, Proc. Natl. Acad. Sci. USA 89:10892-10895; Hwu et al., 1993, J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or U1 snRNA promoter) or preferably RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g. the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Preferably, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the K-Ras dsRNAs bind to target K-Ras RNA and modulate its function or expression. Delivery of K-Ras dsRNA expressing vectors can be systemic, such as by intravenous or intramuscular administration, by administration to target cells ex-planted from the patient followed by reintroduction into the patient, or by any other means that allows for introduction into a desired target cell.

K-Ras dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single target gene or multiple target genes over a period of a week or more are also contemplated by the present invention. Successful introduction of the vectors of the invention into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The K-Ras dsRNA molecules can also be inserted into vectors and used as gene therapy vectors for human pancreatic cancer patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Example 4

### Method of Determining an Effective Dose of a K-Ras dsRNA

A therapeutically effective amount of a composition containing a sequence that encodes K-Ras specific dsRNA, (i.e., an effective dosage), is an amount that inhibits expression of the polypeptide encoded by the K-Ras target gene by at least 10 percent. Higher percentages of inhibition, e.g., 15, 20, 30, 40, 50, 75, 85, 90 percent or higher may be preferred in certain embodiments. Exemplary doses include milligram or microgram amounts of the molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). The compositions can be administered one time per week for between about 1 to 1 0 weeks, e.g., between 2 to 8 weeks, or between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. In some cases transient expression of the dsRNA may be desired. When an inducible promoter is included in the construct encoding an dsRNA, expression is assayed upon delivery to the subject of an appropriate dose of the substance used to induce expression.

Appropriate doses of a composition depend upon the potency of the molecule (the sequence encoding the dsRNA) with respect to the expression or activity to be modulated. One or more of these molecules can be administered to an animal (e.g., a human) to modulate expression or activity of one or more target polypeptides. A physician may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The efficacy of treatment can be monitored either by measuring the amount of the K-Rastarget gene mRNA (e.g. using real time PCR) or the amount of polypeptide encoded by the target gene mRNA (Western blot analysis). In addition, the attending physician will monitor the symptoms associated with pancreatic cancer afflicting the patient and compare with those symptoms recorded prior to the initiation of dsRNA treatment.

We claim:

1. A double-stranded ribonucleic acid (dsRNA) for inhibiting the expression of a K-ras oncogene in a cell, wherein the dsRNA comprises a complementary RNA strand comprising a nucleotide sequence which is complementary to at least a part of the K-ras oncogene.

2. The dsRNA of claim 1, further comprising a sense RNA strand, and wherein at least one of said RNA strands comprises a nucleotide overhang of 1 to 4 nucleotides in length.

3. The dsRNA of claim 2, wherein the nucleotide overhang is 2 or 3 nucleotides in length.

4. The dsRNA of claim 2, wherein the nucleotide overhang is on a 3′-terminus of the complementary RNA strand.

5. The dsRNA of claim 4, wherein the complementary RNA strand comprises a 5′-end, and wherein the 5′-end is blunt.

6. The dsRNA of claim 1, wherein the K-ras oncogene is a K-ras gene comprising a point mutation in codon 12.

7. The dsRNA of claim 6, wherein codon 12 encodes an amino acid selected from the group consisting of arginine, serine, alanine, valine, cystein, and asparagine.

8. The dsRNA of claim 1, wherein the K-ras oncogene is a K-ras gene comprising a point mutation in codon 13.

9. The dsRNA of claim 8, wherein codon 13 encodes asparagine.

10. The dsRNA of claim 1, wherein the K-ras oncogene is a K-ras gene comprising a point mutation in codon 61.

11. The dsRNA of claim 10, wherein codon 61 encodes histidine or leucine.

12. The dsRNA of claim 1, wherein the nucleotide sequence is less than 25 nucleotides in length.

13. The dsRNA of claim 1, wherein the nucleotide sequence is 19 to 24 nucleotides in length.

14. The dsRNA of claim 1, wherein the nucleotide sequence is 20 to 24 nucleotides in length.

15. The dsRNA of claim 1, wherein the nucleotide sequence is 21 to 23 nucleotides in length.

16. The dsRNA of claim 1, wherein the nucleotide sequence is 22 or 23 nucleotides in length.

17. The dsRNA of claim 1, wherein the complementary RNA strand is less than 30 nucleotides in length.

18. The dsRNA of claim 1, wherein the complementary RNA strand is less 25 nucleotides in length.

19. The dsRNA of claim 1, wherein the complementary RNA strand is 21 to 24 nucleotides in length.

20. The dsRNA of claim 1, wherein the dsRNA further comprises a second (sense) RNA strand.

21. The dsRNA of claim 20, wherein the complementary RNA strand is 23 nucleotides in length and the second RNA strand is 21 nucleotides in length.

22. The dsRNA of claim 21, wherein the complementary RNA strand further comprises a 3′-end and a 5′-end, wherein the 3′-end comprises a nucleotide overhang of 2 nucleotides in length, and wherein the 5′-end is blunt.

23. The dsRNA of claim 1, wherein the nucleotide sequence of the complementary RNA strand is complementary to a primary or processed RNA transcript of the K-ras oncogene.

24. The dsRNA of claim 20, wherein the complementary RNA strand comprises SEQ ID NO:2 and the second RNA strand comprises SEQ ID NO:1.

25. The dsRNA of claim 20, wherein the complementary RNA strand comprises SEQ ID NO:4 and the second RNA strand comprises SEQ ID NO:3.

26. The dsRNA of claim 20, wherein the complementary RNA strand comprises SEQ ID NO:5 and the second RNA strand comprises SEQ ID NO:6.

27. The dsRNA of claim 1, wherein the cell is a pancreatic carcinoma cell.

28. A method for inhibiting the expression of a K-ras oncogene in a cell, the method comprising:
(a) introducing into the cell a double-stranded ribonucleic acid (dsRNA), wherein the dsRNA comprises a complementary RNA strand comprising a nucleotide sequence which is complementary to at least a part of the K-ras oncogene; and
(b) maintaining the cell produced in step (a) for a time sufficient to obtain degradation of a mRNA transcript of the K-ras oncogene, thereby inhibiting expression of the target gene in the cell.

29. The method of claim 28, further comprising a sense RNA strand, and wherein at least one of said RNA strands comprises a nucleotide overhang of 1 to 4 nucleotides in length.

30. The method of claim 28, wherein the nucleotide overhang is 2 or 3 nucleotides in length.

31. The method of claim 28, wherein the nucleotide overhang is on a 3′-terminus of the complementary RNA strand.

32. The method of claim 31, wherein the complementary RNA strand comprises a 5′-end, and wherein the 5′-end is blunt.

33. The method of claim 28, wherein the K-ras oncogene is a K-ras gene comprising a point mutation in codon 12.

34. The method of claim 33, wherein codon 12 encodes an amino acid selected from the group consisting of arginine, serine, alanine, valine, cystein, and asparagine.

35. The method of claim 28, wherein the K-ras oncogene is a K-ras gene comprising a point mutation in codon 13.

36. The method of claim 35, wherein codon 13 encodes asparagine.

37. The method of claim 29, wherein the K-ras oncogene is a K-ras gene comprising a point mutation in codon 61.

38. The method of claim 37, wherein codon 61 encodes histidine or leucine.

39. The method of claim 28, wherein the nucleotide sequence is less than 25 nucleotides in length.

40. The method of claim 28, wherein the nucleotide sequence is 19 to 24 nucleotides in length.

41. The method of claim 28, wherein the nucleotide sequence is 20 to 24 nucleotides in length.

42. The method of claim 28, wherein the nucleotide sequence is 21 to 23 nucleotides in length.

43. The method of claim 28, wherein the nucleotide sequence is 22 or 23 nucleotides in length.

44. The method of claim 28, wherein the complementary RNA strand is less than 30 nucleotides in length.

45. The method of claim 28, wherein the complementary RNA strand is less 25 nucleotides in length.

46. The method of claim 28, wherein the complementary RNA strand is 21 to 24 nucleotides in length.

47. The method of claim 28, wherein the dsRNA further comprises a second (sense) RNA strand.

48. The method of claim 47, wherein the complementary RNA strand is 23 nucleotides in length and the second RNA strand is 21 nucleotides in length.

49. The method of claim 48, wherein the complementary RNA strand further comprises a 3′-end and a 5′-end, wherein the 3′-end comprises a nucleotide overhang of 2 nucleotides in length, and wherein the 5′-end is blunt.

50. The method of claim 28, wherein the nucleotide sequence of the complementary RNA strand is complementary to a primary or processed RNA transcript of the K-ras oncogene.

51. The method of claim 29, wherein the complementary RNA strand comprises SEQ ID NO:2 and the second RNA strand comprises SEQ ID NO:1.

52. The method of claim 29, wherein the complementary RNA strand comprises SEQ ID NO:4 and the second RNA strand comprises SEQ ID NO:3.

53. The method of claim 29, wherein the complementary RNA strand comprises SEQ ID NO:5 and the second RNA strand comprises SEQ ID NO:6.

54. The method of claim 28, wherein the cell is a pancreatic carcinoma cell.

55. A pharmaceutical composition for inhibiting the expression of a K-ras oncogene in a mammal, comprising a dsRNA and a pharmaceutically acceptable carrier, wherein the dsRNA comprises a complementary RNA strand comprising a complementary nucleotide sequence which is complementary to at least a part of the K-ras oncogene.

56. The pharmaceutical composition of claim 55, further comprising a sense RNA strand, and wherein at least one of said RNA strands comprises a nucleotide overhang of 1 to 4 nucleotides in length.

57. The pharmaceutical composition of claim 56, wherein the nucleotide overhang is on a 3′-terminus of the complementary RNA strand.

58. The pharmaceutical composition of claim 55, wherein the K-ras oncogene is a K-ras gene comprising a point mutation in codon 12, codon 13, or codon 61.

59. The pharmaceutical composition of claim 55, wherein the nucleotide sequence is less than 25 nucleotides in length.

60. The pharmaceutical composition of claim 55, wherein the nucleotide sequence is 19 to 24 nucleotides in length.

61. The pharmaceutical composition of claim 55, wherein the nucleotide sequence is 20 to 24 nucleotides in length.

62. The pharmaceutical composition of claim 55, wherein the complementary RNA strand is less than 30 nucleotides in length.

63. The pharmaceutical composition of claim 55, wherein the complementary RNA strand is less 25 nucleotides in length.

64. The pharmaceutical composition of claim 55, wherein the dsRNA further comprises a second (sense) RNA strand.

65. The pharmaceutical composition of claim 64, wherein the complementary RNA strand is 23 nucleotides in length and the second RNA strand is 21 nucleotides in length.

66. The pharmaceutical composition of claim 65, wherein the complementary RNA strand further comprises a 3′-end and a 5′-end, wherein the 3′-end comprises a nucleotide overhang of 2 nucleotides in length, and wherein the 5′-end is blunt.

67. The pharmaceutical composition of claim 64, wherein the complementary RNA strand comprises SEQ ID NO:2 and the second RNA strand comprises SEQ ID NO:1.

68. The pharmaceutical composition of claim 64, wherein the complementary RNA strand comprises SEQ ID NO:4 and the second RNA strand comprises SEQ ID NO:3.

69. The pharmaceutical composition of claim 64, wherein the complementary RNA strand comprises SEQ ID NO:5 and the second RNA strand comprises SEQ ID NO:6.

70. The pharmaceutical composition of claim 55, wherein the cell is a pancreatic carcinoma cell.

71. The pharmaceutical composition of claim 70, wherein the organism is a mammal.

72. The pharmaceutical composition of claim 71, wherein the mammal is a human.

73. The pharmaceutical composition of claim 55, wherein the dosage unit of dsRNA is less than 5 milligram (mg) of dsRNA per kg body weight of the mammal.

74. The pharmaceutical composition of claim 55, wherein the dosage unit of dsRNA is in a range of 0.01 to 2.5 milligrams (mg), 0.1 to 200 micrograms (μg), 0.1 to 100 μg per kilogram body weight of the mammal.

75. The pharmaceutical composition of claim 55, wherein the dosage unit of dsRNA is less than 25 μg per kilogram body weight of the mammal.

76. The pharmaceutical composition of claim 55, wherein the pharmaceutically acceptable carrier is an aqueous solution.

77. The pharmaceutical composition of claim 76, wherein the aqueous solution is phosphate buffered saline.

78. The pharmaceutical composition of claim 55, wherein the pharmaceutically acceptable carrier comprises a micellar structure selected from the group consisting of a liposome, capsid, capsoid, polymeric nanocapsule, and polymeric microcapsule.

79. The pharmaceutical composition of claim 78, wherein the micellar structure is a liposome.

80. A method for treating a disease caused by the expression of a K-ras oncogene in a mammal, which comprises administering to said mammal a pharmaceutical composition comprising a double-stranded ribonucleic acid (dsRNA) and a pharmaceutically acceptable carrier, wherein the dsRNA comprises a complementary RNA strand comprising a complementary nucleotide sequence which is complementary to at least a part of the K-ras oncogene.


SEQUENCE LISTING (8 sequences)

SEQ ID NO: 1 - RNA, 21 nt, Homo sapiens
aguuggagcuguuggcguagg

SEQ ID NO: 2 - RNA, 23 nt, Homo sapiens
ccuacgccaacagcuccaacuac

SEQ ID NO: 3 - RNA, 21 nt, Homo sapiens
aguuggagcugauggcguagg

SEQ ID NO: 4 - RNA, 23 nt, Homo sapiens
ccuacgccaucagcuccaacuac

SEQ ID NO: 5 - RNA, 21 nt, Homo sapiens
aguuggagcugguggcguagg

SEQ ID NO: 6 - RNA, 23 nt, Homo sapiens
ccuacgccaccagcuccaacuac

SEQ ID NO: 7 - RNA, 22 nt, Synthetic sequence
  Description of the synthetic sequence sense strand of a dsRNA that is complementary to a sequence of the neomycin resistance gene
caaggaugaggaucguuucgca

SEQ ID NO: 8 - RNA, 23 nt, Synthetic sequence
  Description of the synthetic sequence antisense strand of a dsRNA that is complementary to a sequence of the neomycin resistance gene
gcgaaacgauccucauccugucu